(19) United States
(12) Patent Application Publication
(10) Pub. No.: US 2013/0312766 A1
(43) Pub. Date: Nov. 28, 2013

(54) Breast Compression Band

(71) Applicant: Suzette R. Duggan, Allenwood, NJ (US)

(72) Inventor: Suzette R. Duggan, Allenwood, NJ (US)

(21) Appl. No.: 13/901,655

(22) Filed: May 24, 2013

Related U.S. Application Data
(60) Provisional application No. 61/651,223, filed on May 24, 2012.

(51) Int. Cl. A61B 19/00

(52) U.S. Cl. CPC A61B 19/00
USPC 128/845

(57) ABSTRACT

A compression band for at least one breast comprises a strap with a pocket integral and one piece therewith filled with a granular material forming a weight of about one to two pounds. The strap and pocket are relatively inelastic fabrics that are soft to the touch such as cotton, polyester velour, flannel or fleece. The strap and pocket are formed from a single sheet of fabric and sewn at seams to form the strap and pocket. One pocket may be dimensioned for a single or both breasts, the strap being attached about the torso by Velcro type fasteners. The weight induced by the force of gravity provides the desired compression.

This invention relates to post operative breast compression bands. Applicant claims priority on provisional application Ser. No. 61/651,223 filed May 24, 2012, incorporated by reference herein in its entirety.

Post operative surgery for insertion of breast implants typically use a compression band to displace implants to their desired position and hold the implants in place during healing. The prior art compression band is an elastic relatively durable fabric that encircles the woman's body at the upper breast region overlapping a portion of the breasts at their upper regions. This prior art band is about 3-4 inches wide and relative thin of approximately one or two layers of the fabric. The ends of the band have commercial hook and loop fasteners (for example, Velcro brand fasteners) for attaching them together to form a stretched elastic loop about the body. The stretched loop provides the desired compression on the breasts to keep the implants in place during healing.

Such a band is normally kept on the patient for about two or three months. However, this band can be uncomfortable and can even be painful to the patient due to the relative high forces being exerted on the body throughout the band's periphery due to the fact that the band is elastic and so exhibits radial compression forces throughout its length. Some of such periphery, for example, under the arm pits and even the breasts themselves, experience high discomfort due to the nature of the band's construction and fabric used. The present inventor recognizes a need to overcome the problems with this prior art compression band.

A further breast compression device is disclosed in U.S. Pat. No. 6,540,702 to Maria Sarango which discloses a harness and a breast compression member. This is believed to be uncomfortable to the wearer by the present inventor. Further, it is relatively complex and costly since it employs compression pistons, sensors, an lair pressure pump, a pressure gauge and a power source.

U.S. Pub. No. 2011/03192235 to James Garrett discloses a sports training belt. This is worn about the waist. A plurality of weighted tubes are included, each are sealed with a filler material such as lead pellets, each comprising two pound, four pound and six pounds and a flexible. This device is not suitable for medical related breast compression as the weight values are too high and the device is too bulky to fit under the arm pits of a person. Also for breast compression, the weights extend for about three fourths a person's circumference and thus would not be appropriate for breast compression due to its excessive weighted region length. Also for breast compression the device would have to fit just beneath the arm pits which appear to leave little or no room for this device at this region due to the presence of the weights.

Of interest is U.S. Pat. No. 7,090,558 directed to a weighted sports bra and U.S. Pat. No. 4,1394,012 directed to a weighted exercise vest. Neither of these patents disclose a device useful for breast compression with respect to medical applications.

The present inventor recognizes a need for a medical related breast compression device that is comfortable for the wearer, is not obtrusive painful or otherwise induces discomfort to the user as does the present devices discussed above and need not be worn 24 hours a day for successive days in a scheduled period also as required by the prior art device.

A breast compression band according to an embodiment of the present invention comprises an elongated flexible substrate of a length for encircling a human body at the chest region beneath the arm pits for engaging the female breasts. A layer of a second given material forms an enclosed pocket with the substrate for juxtaposition with at least one of the breasts when the substrate is attached to the body. A filler material is inside the pocket having an approximate weight of at least about one pound, and an arrangement is included for releasably securing the substrate with the enclosed pocket juxtaposed and engaged with at least one breast to thereby compress the at least one breast by gravitational force.

In a further embodiment, the substrate comprise a fabric.

In a further embodiment, the substrate is a fabric with a relatively soft nap for facing the skin of the encircled body.

In a further embodiment, the substrate comprises at least one of a velour, a fleece, a flannel or any other relatively non-irritating fabric material with respect to the body's skin including cotton, nylon or polyester.

In a further embodiment, the enclosed pocket has a length of at least about four to seven inches and a width in the range of about one to three inches.

In a further embodiment, the enclosed pocket has a length of at least about four inches to twelve inches, a width in the range of about one to three inches, and a weight of about one to two pounds.

In a further embodiment, the substrate is substantially inelastic.

A breast compression band according to a still further embodiment comprises an elongated flexible substrate of a given material for encircling a human body at the chest region beneath for engaging the female breasts. A portion of the substrate forms an enclosed pocket for juxtaposition with at least one of the breasts when the substrate is attached to the body. A filler material is inside the pocket having an approximate weight of at least about one pound. An arrangement releasably secures the substrate with the enclosed pocket engaged with the at least one breast to thereby compress that at least one breast by gravitational force.

A breast compression band according to a still further embodiment of the present invention comprises an elongated pocket, the pocket being sufficiently long and wide to engage the at least one female human breast, the pocket being filled with a material having a weight of at least one pound for compressing the at least one breast; and
a strap attached to the pocket for encircling and being secured to a human body at the chest region with the pocket overlapping at least a portion of the at least one human breast.

### IN THE DRAWING

FIG. 1 is a front elevation view of the breast compression band of one embodiment of the present invention attached to a person;

FIG. 2 is a plan view of the band of FIG. 1 in its unattached state;

FIG. 3 is a top plan perspective view of the band of FIG. 1;

FIG. 4 is a side elevation view of the device according a further embodiment of the present invention;

FIG. 5 is a side elevation view of a further embodiment of the present invention;

FIG. 6 is a sectional end elevation view of the embodiment of FIG. 2 taken along lines 6-6;

FIG. 7 is a side elevation view of a portion of the strap of FIG. 2 taken along lines 7-7; and

FIG. 8 is a plan view of a portion of the band of FIG. 2 corresponding to region 8 of FIG. 2, but depicting a further embodiment of the band.

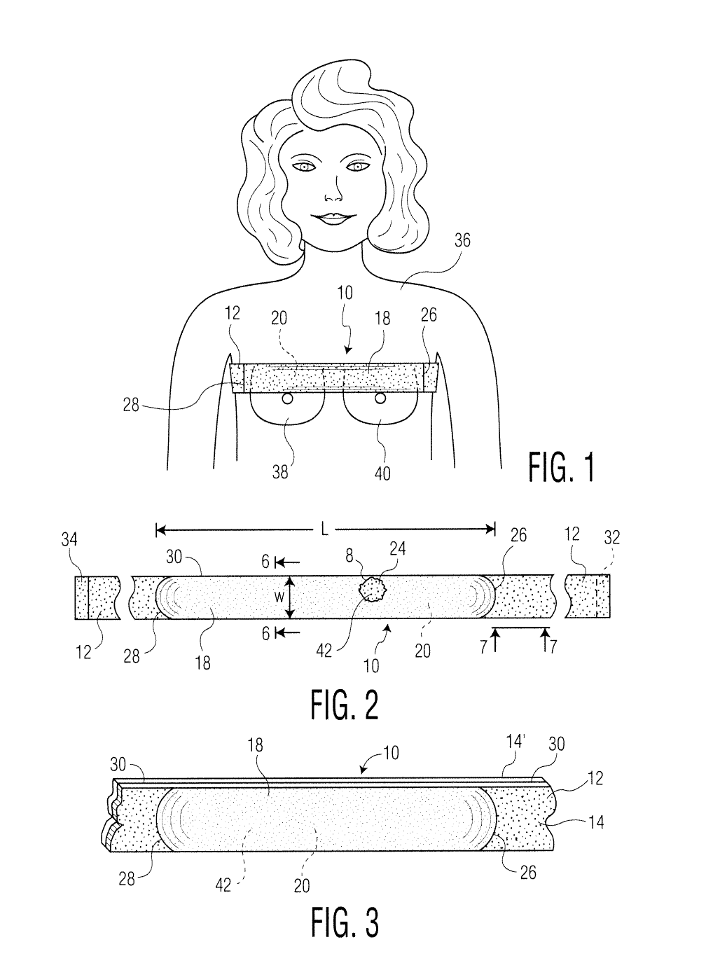

In FIG. 1, breast compression band 10 according to one embodiment of the present invention is intended for therapeutic treatment of one or both breasts of a postoperative female patient that has undergone breast augmentation with a breast implant or breast reconstruction following a mastectomy. The band 10 can be used to provide compression to one or both breasts according to a given implementation.

The band 10 in the embodiment of FIGS. 1-3 preferably in this embodiment comprises a fabric 12, such as velour, polyester fleece or other material forming a fleece, cotton flannel, cotton, a knitted or woven fabric, a nylon fabric, a felt, or lace, percales or any similar other fabric or material that is relatively soft to the touch and not irritating to the skin. For example, muslin or denim are not considered to be as desirable as they may be an irritant to skin. The fabric being woven or knitted, is not normally deemed elastic and is believed to be generally inelastic.

Woven and knitted fabrics, due to their weaves, might exhibit some elasticity, that is not intended to mean inelastic as in the case of rubber or other elastomeric material that readily expands in response to an applied force and immediately returns to its quiescent state with an applicable force when the force is removed. Knitted and woven fabrics do not normally exhibit such relatively high resiliency and thus are preferred.

Elasticity as provided by an elastomeric material is not desired as such material when stretched applies radial forces when wrapped about the human body throughout the material. Such radial forces are not desired as they can cause discomfort to the wearer of the band at certain sensitive regions, especially under the armpits or other regions of the body which might be more sensitive to such applied forces.

The band 10 in this embodiment is preferably a one piece fabric such as of the type noted above sectioned to form the different regions of the band 10. The band 10 may, however, comprise multiple pieces of fabric or other material, which pieces may be the same or different for the different portions of the band 10. Preferably a napped fabric such as cotton velour is desired at least for the portion that faces and is contiguous with the skin. Any fabric or material that faces and is contiguous with the skin should be non-irritating to the skin. The other portions of the band 10 may comprise any other fabric or material as desired. The fabric or other material is in sheet form and formed into preferably a two layer strap 12 that has a pile or nap 16, FIG. 7. The pile or nap 16 faces outwardly of the strap in opposite directions. In FIG. 2, the nap faces outwardly toward the reader and inwardly into the drawing and formed of two juxtaposed layers 14, 14′.

The layer 14 of the fabric at the central portion 18 of the band 10, FIGS. 1,2 and 3, forms a pocket 20, FIG. 6 by being wrapped so that the two layers 14, 14′ are juxtaposed but spaced apart to form an interior space 24 of the pocket, FIG. 6. The layers 14, 14′ are joined at side seam 22, FIG. 6, to fully enclose the pocket 20. The upper layer 14 is stitched to the bottom layer 14′ at transverse end seams 26, 28 and along side seam 30 to form the pocket 20. The side seam 30 attaches the upper and lower layers 14, 14′ to form the strap 12 at each end of the pocket 20.

Attachment devices, such as hook fastener 32 and loop fastener 34, e.g., Velcro brand type fasteners, are attached to opposite ends of the pocket 20 for securing the band 10 to encircle the person 36. The pocket 20 in this embodiment is a single pocket that engages both breasts 38, 40 at their upper portions thereof. This position provides compressive downward forces on each breast upper region. These breast have previously received corresponding implants.

The single pocket 20 is filled with a filler 42 that provides a weight of about two pounds in the case of two breasts being compressed by the pocket 20. The filler 42 preferably comprises any granular material, such as fine sand, and the like. The sand is preferably fine, i.e., relatively small grains to provide a smooth, fine soft like feel to the body of the person wearing the band 10. Course grains tend to be bumpy and thus may be slightly more uncomfortable to the wearer. The filler may be any other material that provides the desired weight in the given space defined by the pocket 20. Such filler material may includes spheres (or other objects) of any size, but preferably as small as possible to minimize undesirable bumps in the exterior of the pocket. The filler may be fibrous as well as granular. It may be metals, sand, or any other material that can provide the desired weight in the given space of the pocket.

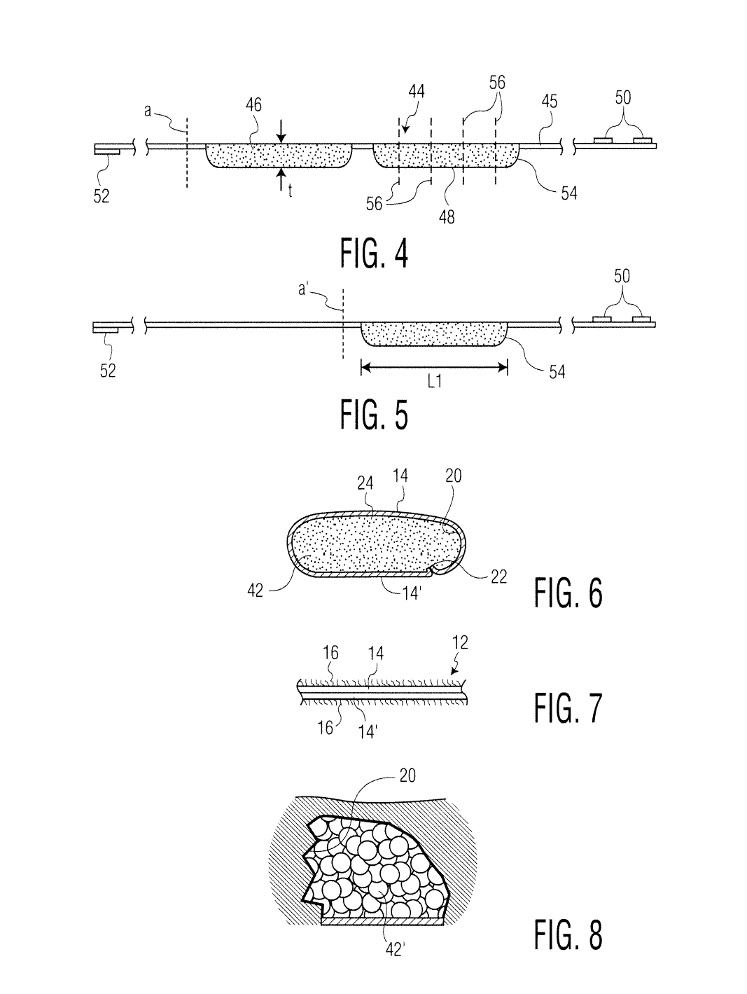

In this embodiment, the single pocket 20 engaged with two breasts to be compressed is preferably about 9-14 inches in the length direction L, FIG. 2. The strap 12 and pocket 20 are preferably about 2-3 inches wide, dimension w, FIG. 2, but could be narrower, e.g., 1 inch or wider, to about 3-5 inches. The pocket 20 is about 1 to 1 ½ inches deep, dimension t, FIG. 4, normal to the drawing sheet, FIGS. 1 and 2. Of course, the size of the pocket may differ from these dimensions according to the desired weight of the filler 42 and proportions of the wearer. The strap 12, being one or two layers of fabric, is about 118^{th} inch to about ¼ inch thick according to the fabric employed.

While the strap and pocket are formed from the same material, it is contemplated that the pocket and strap may be formed of different materials, e.g., velour or other fabric, polyester, cotton, nylon, for example, for the pocket and lace or other decorative materials for the strap, and so on.

In FIG. 4, a further embodiment, illustrates a compression band 44 which comprises two spaced pockets 46, 48, each associated with a different breast. These pockets are each about 6-8 inches in length L1, FIG. 5 (illustrating a pocket 54 for one breast compression). The other relative dimensions of the strap 45 and pockets 46, 48, for the embodiment of FIG. 4, are approximately the same as the corresponding dimensions for the embodiment described for FIGS. 1-3. In this embodiment, the strap 45 has a series of spaced fasteners, e.g., loops or hooks fasteners 50 at one strap end and the mating fastener(s) 52 at the other strap end for adjusting the length of the attached band to the corresponding dimensions of the wearer. The length L1 of a single pocket 46, 48 (FIGS. 4) and 54 (FIG. 5) is preferably about five to eight inches, but can be other values according to a given implementation.

In FIG. 8, a filler 42′ for a pocket such as pocket 20 is illustrated as spheres which could be any material such as a metal or plastic, The spheres are enlarged relative to the pocket for purposes of illustration, but in practice may be microspheres similar to fine grains of sand or powder, to avoid lumpiness in the exterior surface to avoid discomfort to the user.

In FIG. 5 a single pocket 54 would be used for a person with a need for compression on only one breast. It should be understood that the strap, such as strap 45, FIG. 4 may be any desired length as illustrated by the dashed line a, which represents a foreshortened strap 45. This is also shown in FIG. 5 by the dashed line a′.

While the various pockets 46, 48 and 54 are illustrated as comprising a space having single volume, the pockets could be made with a series of separate spaces or compartments, which may be filled with the same or different materials as might be desired in a given implementation. Such compartments would be implemented with seams represented by the dashed lines 56, FIG. 4, relative to pocket 48.

In operation, the band is wrapped about the torso at the chest area engaged with the upper portion of the applicable breast. The weight of the band 10, by the force of gravity, forces the implant inside a breast lower in the breast cavity and holds the implant in place during healing. The disclosed band is comfortable to the user as compared to prior art elastic bands without such weight. The weight provides the desired compression by the force of gravity instead of the radial forces exerted by the prior art elastic band. The wearer lies on her back during sleep periods, e.g., 6-8 hours, to obtain the benefit of the band. Experience has shown the band as disclosed herein is effective after a few weeks of use as compared to several months of continued round the clock recommended use with a prior art elastic band, which is extremely uncomfortable.

It will occur to one of ordinary skill that various modifications may be made to the disclosed embodiment, it being intended that the scope of the invention be defined by the appended claims.

What is claimed is:

1. A female breast compression band comprising:
an elongated flexible substrate of at least a first given material having a length for encircling a human body at the chest region beneath the arm pits engaged with the female breasts;
a layer of at least a second given material forming an enclosed pocket with the substrate located on the substrate for juxtaposition with at least one of said breasts when the substrate is attached to the body;
a filler material inside the pocket having an approximate weight of at least approximately one pound; and
an arrangement for releasably securing the substrate with the enclosed pocket juxtaposed and engaged with at least one of the breasts to thereby compress the at least one breast by gravitational force.

2. The compression band of claim 1 wherein the substrate comprise a fabric.

3. The compression band of claim 1 wherein the substrate is a fabric with a relatively soft non-irritating surface for facing and engaging the skin of the encircled body.

4. The compression band of claim 1 wherein the substrate is a fabric with a relatively soft surface in at least a portion thereof for at least facing the skin of the encircled body, the fabric comprising at least one of a velour, a fleece, a flannel or any other relatively skin non-irritating material including cotton, nylon or polyester.

5. The compression band of claim 1 wherein the enclosed pocket has a length of at least about four to seven inches and a width in the range of about one to three inches.

6. The compression band of claim 1 wherein the enclosed pocket has a length of at least about four inches to twelve inches, a width in the range of about one to three inches, a depth of about 1% inches and a weight of about one to two pounds.

7. The compression band of claim 1 wherein the substrate is substantially inelastic.

8. The compression band of claim 1 wherein the filler material is at least one of granular, fibrous or spheres.

9. A breast compression band comprising:
an elongated flexible substrate of a given material and a length for encircling a human body at the chest region beneath the arm pits for engaging the upper region of the female breasts;
a portion of the substrate forming an enclosed pocket for juxtaposition with at least one of said breasts when the substrate is attached to the body;
a filler material inside the pocket having an approximate weight of at least about one pound; and
an arrangement for releasably securing the substrate with the enclosed pocket engaged with said at least one breast to thereby compress that at least one breast by gravitational force.

10. The compression band of claim 8 wherein the substrate is substantially inelastic.

11. The compression band of claim 1 wherein the substrate comprise a fabric.

12. The compression band of claim 1 wherein the substrate is a fabric with a relatively soft nap for facing the skin of the encircled body.

13. The compression band of claim 1 wherein the substrate is a fabric comprising at least one of a velour, a fleece, a flannel or any other relatively skin non-irritating material including cotton, nylon or polyester.

14. The compression band of claim 1 wherein the enclosed pocket has a length of at least about four to seven inches and a width in the range of about one to three inches.

15. The compression band of claim 1 wherein the enclosed pocket has a length of at least about four inches to twelve inches, a width in the range of about one to three inches, and a weight of about one to two pounds.

16. A breast compression band comprising:
an elongated pocket of a given length, the pocket being sufficiently long and wide to engage the upper region of at least one female human breast, the pocket being filled with a material having a weight of at least one pound for compressing the at least one breast; and
a strap attached to the pocket for encircling and being secured to a human body at the chest region with the pocket overlapping at least a portion of the at least one human breast.

17. The compression band of claim 16 wherein the substrate comprise a relatively inelastic fabric.

18. The compression band of claim 16 wherein the substrate is a fabric with a relatively soft nap for facing the skin of the encircled body.

19. The compression band of claim 16 wherein the substrate is a fabric comprising at least one of a velour, a fleece, a flannel or any other skin relatively non-irritating material including cotton, nylon or polyester.

20. The compression band of claim 16 wherein the enclosed pocket has a length of at least about four to seven inches and a width in the range of about one to three inches.

21. The compression band of claim 16 wherein the enclosed pocket has a length of at least about four inches to twelve inches, a width in the range of about one to three inches, and a weight of about one to two pounds.

22. The compression band of claim 16 wherein the filler material is at least one of granular, fibrous or spheres.
